(19) United States
(12) Patent Application Publication
(10) Pub. No.: US 2018/0303576 A1
(43) Pub. Date: Oct. 25, 2018

(54) APPARATUSES, METHODS, AND SYSTEMS FOR THE IDENTIFICATION AND TREATMENT OF PULMONARY TISSUE

(71) Applicant: Spiration, Inc. d/b/a Olympus Respiratory America, Redmond, WA (US)

(72) Inventors: David H. Dillard, Grapeview, WA (US); Hugo X. Gonzalez, Woodinville, WA (US); Peter D. Hoffman, Pittsburgh, PA (US); Brandon J. Shuman, Kirkland, WA (US)

(21) Appl. No.: 16/024,144

(22) Filed: Jun. 29, 2018

Related U.S. Application Data
(63) Continuation of application No. 14/498,365, filed on Sep. 26, 2014.
Application No. 14/498,365 is a continuation of application No. PCT/US2013/031067, filed on Mar. 13, 2013.
(60) Provisional application No. 61/617,590, filed on Mar. 29, 2012.

(51) Int. Cl. A61B 90/70; A61B 5/05; A61B 5/055; A61B 5/06; A61B 17/34; A61B 90/00; A61B 18/14; A61B 18/04; A61B 18/00; A61N 7/02

(52) U.S. Cl. CPC A61B 90/70; A61B 2018/00577; A61B 5/055; A61B 5/062; A61B 17/3468; A61B 90/39; A61B 18/1492; A61B 2090/3966; A61B 2090/3954; A61B 2090/392; A61B 2090/3908; A61B 2090/3995; A61B 2562/17; A61B 18/04; A61B 2018/00047; A61B 2018/00541; A61N 7/022; A61B 5/05

(57) ABSTRACT

Devices, systems, and methods for implanting and locating traceable markers in a region of a patient's body such as a lung, and in particular lung nodules which may be difficult to locate using traditional means. Further embodiments describe devices, systems, and methods that may be used to treat regions in the lung such as lung nodules with various treatment modalities including heating, microwave irradiation, chemical treatment, and which may be used in conjunction with embodiments of the traceable markers described herein.

### CROSS-REFERENCE TO RELATED APPLICATIONS

This application claims the benefit under 35 U.S.C. § 120 as a continuation of U.S. patent application Ser. No. 14/498,365, titled APPARATUSES, METHODS, AND SYSTEMS FOR THE IDENTIFICATION AND TREATMENT OF PULMONARY TISSUE, filed Sep. 26, 2014, which claims the benefit under 35 U.S.C. § 120 and 35 U.S.C. § 365(c) as a continuation of International Application No. PCT/US2013/031067, designating the United States, with an international filing date of Mar. 13, 2013, titled APPARATUSES, METHODS, AND SYSTEMS FOR THE IDENTIFICATION AND TREATMENT OF PULMONARY TISSUE, which claims the benefit of U.S. Provisional Application No. 61/617,590, titled APPARATUSES, METHODS, AND SYSTEMS FOR THE IDENTIFICATION AND TREATMENT OF PULMONARY TISSUE, filed Mar. 29, 2012, which is hereby incorporated by reference herein in its entirety. Any and all priority claims identified in the Application Data Sheet, or any correction thereto, are hereby incorporated by reference under 37 CFR 1. 57.

### BACKGROUND

### Technical Field

Embodiments of the present disclosure relate generally to the field of medical devices, and in particular, to apparatuses, methods, and devices for identifying and treating portions of the body. In particular, certain embodiments of the present disclosure are related to the identification and treatment of diseased and/or cancerous regions in a lung, and in particular lung nodules.

### Description of the Related Art

Lung cancer has a high incidence of morbidity and mortality in patients. There is presently an extensive effort to develop diagnostic methods for early identification of areas of the lung, for example pulmonary nodules, which may be precursors of cancer. Pulmonary or lung nodules are small masses of tissue in the lung that may range in size between 0.5-30 mm. Pulmonary nodules often require careful evaluation by a medical professional, especially in patients that have risk factors such as tobacco use or a family history of cancer. Imaging methods are evolving to produce more accurate identification of pulmonary nodules to determine whether these may be cancerous or otherwise diseased.

Management of pulmonary nodules has varied from simple observation and follow up at given time intervals to immediate biopsy and surgery. Needle biopsies are often an important step in the management of pulmonary nodules as well as for bigger tumors and/or lung masses. A positive identification of cancer is typically an indication for pulmonary surgery, i.e., lobectomy. Unfortunately, a negative biopsy does not entirely eliminate the risk of cancer at a particular location, and may require further follow up and surgical biopsy.

Although advances in diagnostic methods are in some cases improving the early identification and follow up to lung nodules, the methods for their removal are lagging in development. Biopsy surgery typically consists of a mini-thoracotomy or the use of thoracoscopic or endoscopic methods to access the thoracic cavity and lung tissue. A bronchial blockade is sometimes needed to isolate and deflate the lung segment or lobe where the biopsy will take place. With lung deflation, the anatomy of the lung becomes distorted, making the imaging and anatomical correlations needed to locate the appropriate lung area imprecise, vague, and in some cases useless.

Once the surgeon accesses the deflated lung portion thought to contain the nodule, visual identification is typically used to find the nodule. This task is far easier if the lung area is superficial and accessible. In the case of an isolated lung area, such as a nodule that is small and located deeply within the parenchyma, visual identification may be difficult or impossible, and the surgeon may be forced, for example, to use his or her fingers to palpate the suspected area. The palpation process may thus require performing a mini-thoracotomy (or extending an existing incision) to permit the surgeon's fingers to reach the targeted lung area. Even with palpation, nodules may be difficult to identify.

After the lung area or nodule has been located and identified, surgical removal of the affected area typically follows. Removal may comprise at least a wedge resection of the affected area. If a lung nodule, tumor, or mass is cancerous, current knowledge and recommendations include performing a lobectomy.

### SUMMARY

It is therefore a goal of the embodiments described herein to provide new devices, systems, and methods for the identification and treatment of tissue, in particular lung tissue and lung nodules.

A system for locating a region of tissue can comprise: a traceable marker configured to be implanted and retained in the region of tissue, wherein the traceable marker comprises one or more localization attributes; and, a sensor configured to detect one or more localization attributes. In some embodiments, the system further comprises an insertion instrument configured to implant the traceable marker in the region of tissue. According to some configurations, the localization attribute is magnetic, radioactive, and/or radioopaque. The sensor can further comprises a navigational aid. In some embodiments, the navigation aid comprises a gauge and/or a graphical readout. The sensor can be configured to be inserted through a patient's thoracic cavity and/or into a patient's airway. In some embodiments, the system further comprises a resection device. According to some configurations, the sensor is integrated into the resection device. The resection device can comprise a receptacle configured to aspirate and receive a portion of tissue. In some embodiments, the resection device comprises an elongated, hollow cylindrical body with an aperture at a distal end of the hollow cylindrical body and a conduit connected to a proximal end of the hollow cylindrical body, the conduit being configured to be connected to a source of vacuum. According to some variants, the system further comprises a treatment device. In some embodiments, the traceable marker comprises an auxiliary power lead. The auxiliary power lead can be configured to be selectively connected to a catheter. In some embodiments, the auxiliary power lead is configured to be connected to a secondary module. The secondary module can comprise a power storage module. In some embodiments, the power storage module is configured to be charged wirelessly. The treatment device can be configured to heat the traceable marker electrically. In some embodiments, the treatment device is configured to cause the traceable marker to release an agent from the traceable marker. In some embodiments, the treatment device is configured to heat the traceable marker using magnetic coupling. According to some variants, the treatment device comprises a magnetic probe. The magnetic probe can be attached to a catheter inserted into a patient's airway. In some embodiments, the treatment device is an MRI device. According to some variants, the traceable marker comprises a power source. The power source can comprise a battery and/or a capacitor. In some embodiments, the system further comprises a second traceable marker.

According to some variants, a system for the treatment of a region of tissue can comprise a catheter configured to be inserted within a patient's airway, wherein the catheter comprises an antenna at a distal end of the catheter; a microwave generator configured to be connected to the catheter so as to emit microwave radiation from the antenna. In some embodiments, the system can further comprise a bronchoscope, wherein the catheter is configured for insertion into a working channel of the bronchoscope. The antenna tip can be configured to circumscribe an exterior perimeter of a lung nodule. In some embodiments, the antenna tip is configured to be inserted into a lung nodule. According to some variants, the system further comprises a second antenna tip at the distal end of the catheter.

A method of repeatedly locating a position m a lung can comprise: navigating to the position within the lung; implanting a traceable marker into the position, wherein the traceable marker comprises at least one localization attribute; locating the traceable marker based on the at least one localization attribute. In some embodiments, the method further comprises implanting a second traceable marker near the position within the lung. According to some variants, the step of navigating comprises using a bronchoscope. In some embodiments, the traceable marker is implanted via a catheter or needle inserted into a working channel in the bronchoscope. The method can further comprise biopsying a tissue sample at or near the position in the lung. In some embodiments, the at least one localization attribute is selected from the group consisting of: radioopacity localization, magnetism localization, radioactivity localization, and visual localization. The step of locating the traceable marker can comprise using a localization device. According to some variants, the localization device comprises a magnetic sensor. In some embodiments, the method further comprises applying treatment to the position in the lung. In some embodiments, the treatment is repeatedly applied to the position in the lung by repeatedly locating the traceable marker. The treatment can comprise resecting lung tissue near or at the position in the lung. According to some variants, the step of resecting lung tissue comprises positioning a receptacle in fluid communication with a source of vacuum near the position in the lung, activating the source of vacuum, suctioning lung tissue into the receptacle, and severing the tissue within the receptacle from the remainder of the lung. The treatment can comprise applying microwave radiation. In some embodiments, the microwave radiation is applied via an antenna tip connected to a source of microwave radiation, the antenna tip being inserted into a catheter. According to some variants, the treatment comprises applying heat. The heating can be applied using a magnetic field acting upon the traceable marker. In some embodiments, the magnetic field is applied via a probe positioned near the traceable marker. The magnetic field can be applied using an MRI device. According to some variants, the treatment comprises applying electrical treatment. In some embodiments, the traceable marker comprises a treatment modality. The traceable marker can be configured to release one or more therapeutic agents. In some embodiments, the traceable marker comprises a power source. The power source can a battery attached to the traceable marker. In some embodiments, the power source is attached to the traceable marker via a wire.

According to some variants, a traceable marker is configured to be implanted into a site in a lung and the marker can comprise at least one localization attribute. The marker can comprise a battery. In some embodiments, the marker comprises an auxiliary power lead. The auxiliary power lead may be attached to a source of power. In some embodiments, the auxiliary power lead functions as a wireless charging lead. According to some variants, the marker comprises a radioactive material. The marker can be constructed at least in part from a magnetically-active material.

### BRIEF DESCRIPTION OF THE DRAWINGS

Various embodiments are depicted in the accompanying drawings for illustrative purposes, and the drawings should in no way be interpreted as limiting the scope of the embodiments. In addition, various features of one or more disclosed embodiments can be combined to form additional embodiments, which are part of this disclosure.

FIGS. 1A-C illustrate a portion of a lung with a nodule and embodiments of devices that may be used to implant and locate a traceable marker.

FIG. 2 illustrates an embodiment of a catheter-based sensor that may be used to locate a traceable marker.

FIGS. 3A-E illustrate embodiments of a device that may be used to locate and resect a lung nodule.

FIG. 4 illustrates an embodiment of a system that may be used to treat a lung nodule with radiation such as microwave radiation.

FIGS. 4A-B illustrate embodiments of a system that may be used to treat a lung nodule using multiple markers.

FIGS. 5A-C illustrate embodiments of antenna tips that may be used to treat a lung nodule with radiation such as microwave radiation.

FIG. 6 illustrates an embodiment of a system using a traceable marker heating a lung nodule using magnetic coupling.

FIGS. 7A-B illustrate embodiments of a traceable marker comprising a tail.

FIG. 8 illustrates an embodiment of a traceable marker comprising a secondary module.

FIG. 9 illustrates an embodiment of a traceable marker comprising a secondary module in conjunction with a wireless system.

### DETAILED DESCRIPTION

Embodiments of an apparatus, system, and method for identification and treatment of regions of the lung, and in particular, pulmonary nodules, tumors and/or lesions will be described with reference to the accompanying figures of one or more embodiments. The terminology used in the description presented herein is not intended to be interpreted in any limited or restrictive manner. Rather, the terminology is simply being utilized in conjunction with a detailed description of embodiments of the systems, methods and related components. Furthermore, embodiments may comprise several novel features, no single one of which is solely responsible for its desirable attributes or is believed to be essential to practicing the inventions herein described.

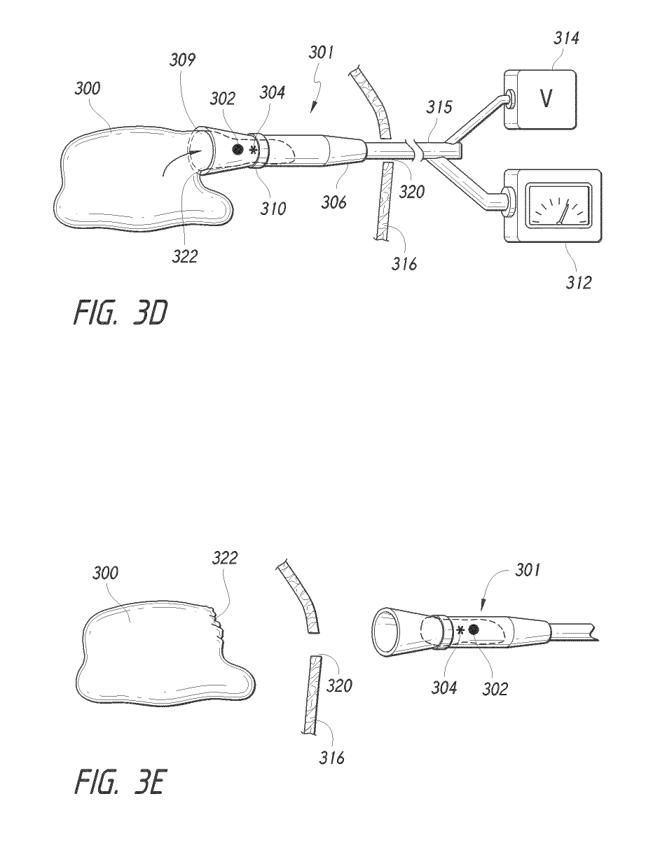

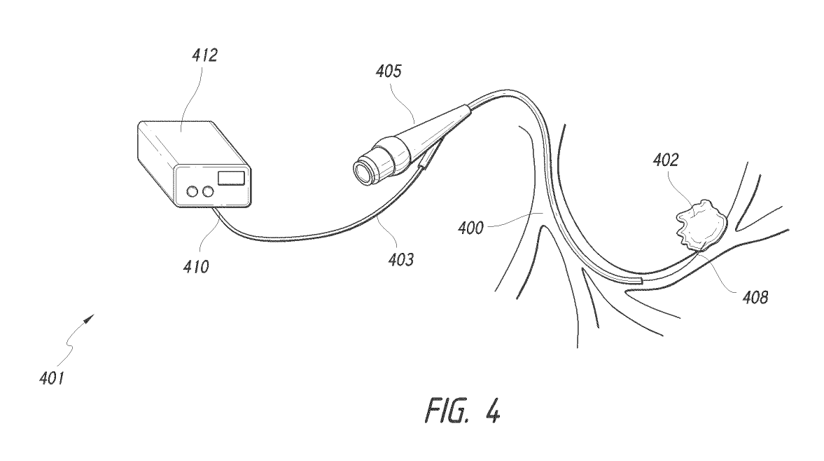

The terms “lung region,” “lung area,” “tissue,” “tumor,” “mass,” and “nodule” as used herein are broad interchangeable terms and, unless otherwise indicated, the terms can include within their meaning, and without limitation, other organs or regions of tissue in a human or animal body, including diseased, cancerous, and/or pre-cancerous tissue, lesions, tumors, masses, or other areas of interest within the body. In general, nodules can be grouped into three or more types. For example, nodules located in the lung parenchyma outside of an airway passage and are not invading or compressing airways (e.g., see nodule 402 of FIG. 4A), some are in the parenchyma and also compress and/or invade the airway (e.g., see nodule 402 of FIG. 4B), and others are primarily located within the airway passage. Although some embodiments described herein refer to identifying and treating an area within a lung, this disclosure is not so limited, and the embodiments described herein may be used, for example, in other vessels, passages, body cavities, and organs in humans and animals.

Figures IA-C illustrate an embodiment that may be used to implant and locate a traceable marker into lung tissue, such as a lung nodule. FIG. IA schematically illustrates a portion of a lung lobe I 00 with a nodule I 02 of suspicious tissue having previously been identified and that then may be examined, biopsied, and treated by a physician. The nodule I 02 may have been previously identified by any diagnostic means, including but not limited to x-rays, magnetic resonance imaging (MRI), ultrasound, or visualization via a catheter inserted into the airway.

Figure IB illustrates a biopsy needle I 06 being used to biopsy tissue in the region of the nodule I 02 via an aperture I 09. The aperture I 09 can be, for example, a port made during a thoracotomy. Here, the biopsy needle I 06 is illustrated as being inserted through the aperture I 09 in the patient's thoracic wall I 08, but it will be appreciated that any biopsy method may be used, including via biopsy needles navigated to the nodule I 02 via a catheter inserted into an airway. In some embodiments, the biopsy needles may include embodiments described in U.S. patent application Ser. No. 13/777,854, entitled LUNG BIOPSY NEEDLE, filed Feb. 26, 2013, which is hereby incorporated by reference herein in its entirety. Before, during, or after the biopsy needle I 06 is used to sample tissue in the region of the nodule I 02, a traceable marker I 04 may also be inserted into or near the nodule I 02. The traceable marker I 04 is preferably configured to be inserted via the biopsy needle I 06, but it can also be inserted separately using another insertion instrument, for example via a second catheter or needle which may be inserted through the aperture I 09. The traceable marker I 04 can have a generally spherical shape. Preferably, the marker I 04 has surface features (e.g., roughness, anchors, biocompatible materials, or any combination thereof) configured to reduce or eliminate the likelihood of the marker 104 dislodging or otherwise migrating from the location to which the marker 104 is deployed. In some embodiments, the markers 104 have non-spherical shapes.

The traceable marker 104 preferably comprises at least one localization attribute that permits it to be detected with a localization device. The localization attribute refers to any attribute that permits the traceable marker 104 to be identified (e.g., after implantation), and may comprise distinguishable visual, radioopaque, magnetic, and/or radioactive markings or attributes, or any combination thereof. In a preferred embodiment, the traceable marker 104 is constructed at least in part of a metal such as, for example but without limitation, iron oxide or stainless steel, and can be localized at least via visual, tactile, radiographic, and magnetic means. The traceable marker 104 may also be covered or coated at least in part with a biocompatible coating, or may be constructed of an inherently biocompatible material (e.g., polished stainless steel, titanium, polymethylmethacrylate, polytetrafluoroethylene) that minimizes or substantially eliminates immunological and other adverse reactions. This may be of particular interest for a traceable marker configured for long term implantation.

The various traceable markers (e.g., traceable marker 104) described herein may be provided with agents that may cause a beneficial, therapeutic, or diagnostic effect in the body and particularly when implanted into or near a lung nodule, for example as a coating or as a part of the traceable marker. Various types of drugs may be incorporated into the traceable marker. Certain chemicals may also be used to provide, for example, a heating effect. Here, a heating effect could be provided by using an air-activated iron-based exothermic reaction typically used in hand-warmers. The agents referred to herein may include chemicals, drugs, or other agents, either alone or in combination, that cause a beneficial, therapeutic, or diagnostic effect with regard to the nodule 102 and/or tissue surrounding the nodule 102, and may comprise anticancer agents (including chemotherapy agents), anti-inflammatory agents, antimicrobial agents, antiviral agents, contrast-enhancing agents (including MRI contrast agents), tissue growth enhancers (including stem cells), tissue growth inhibitors, radioprotective agents, radioactive materials and agents, and other such agents.

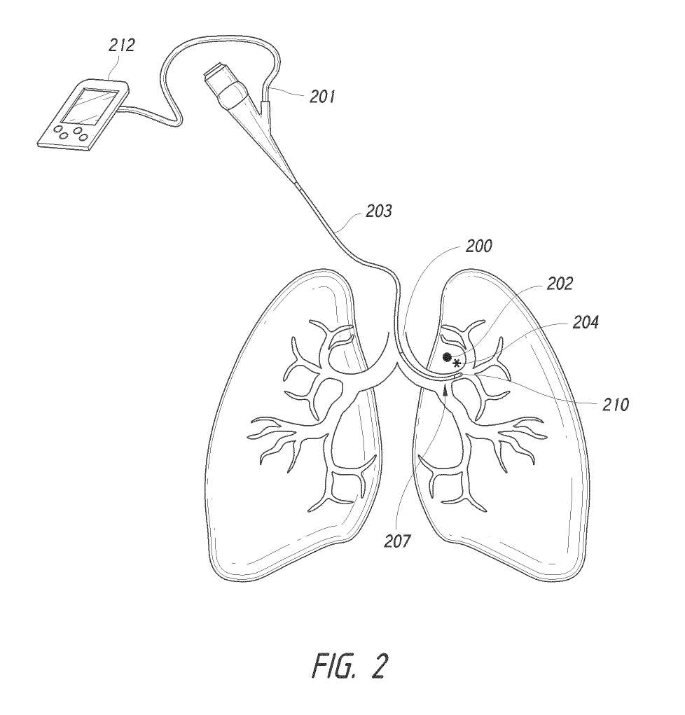

Figure IC illustrates how the marker 104, having been previously implanted in or near the nodule I 02, may be subsequently located. In this embodiment, a sensor 110 responsive to at least one of the localization attributes on the traceable marker I 04 may be navigated near the nodule I 02. The sensor 110 may be brought close to the nodule 102 by insertion into the lung pleural cavity through the aperture 109, although other means may be used. For example, another embodiment may use the sensor 110 inserted or formed into a catheter inserted into the patient's airway, as illustrated in FIG. 2. Because in some embodiments the sensor 110 is preferably configured to not affect the localization attribute or marker 104, such embodiments may permit for repeatedly navigating to and locating the marker 104.

In use, an operator may navigate the sensor 110 in proximity of the nodule 102 by means of a navigation aid 112. For example, the sensor 110 may be configured to identify the marker I 04 by magnetic means, and the sensor 110 may comprise a magnetic induction loop or other such magnetic localization functionality, and the navigation aid 112 may then comprise a gauge illustrating graphically how the magnetic field increases in relation to the proximity and orientation of the sensor 110 with respect to the marker 104. In a preferred embodiment, the navigation aid 112 indicates the direction of the marker I 04 with respect to the location of the sensor 110. Of course, the sensor 110 may be configured to detect any other localization attributes, and could, in the case of the marker I 04 being constructed at least in part from a radioactive material, incorporate a Geiger counter or other sensor responsive to radioactivity. The navigation aid 112 is not necessarily a gauge, and could include, for example, a graphical or numerical readout (e.g., on a computer screen), or could include any one or more of visual, audible, or tactile (e.g., vibrational) feedback responsive to the localization attribute.

FIG. 2 illustrates an embodiment of a catheter-based apparatus that may be combined with other embodiments described herein for the identification and treatment of lung tissue, including, for example, lung nodules. Here, a catheter 20 I may be inserted into a bronchoscope 203 that then inserted into an airway 200 of a patient. The distal end 207 of the catheter 20 I may be provided with a sensor 210 of the type described above in relation to Figures IA-C configured to respond to one or more localization attributes present on an implanted traceable marker 204 implanted in proximity to a nodule 202. In order to aid navigation of the catheter 201 to the location of the marker 204, the sensor 210 may also use a navigation aid 212. Here, the navigation aid 212 is a handheld device, such as, for example, a portable computer, and which may represent graphically how close the sensor 210 is to the marker 204. As such, the navigation aid 212 will aid in navigating to and locating the marker 204. In one embodiment, the navigation aid 212 may depict a map showing all or at least part of the patient's airway 200 in relation to the catheter 201, in particular the distal end 207 of the catheter and/or the sensor 210.

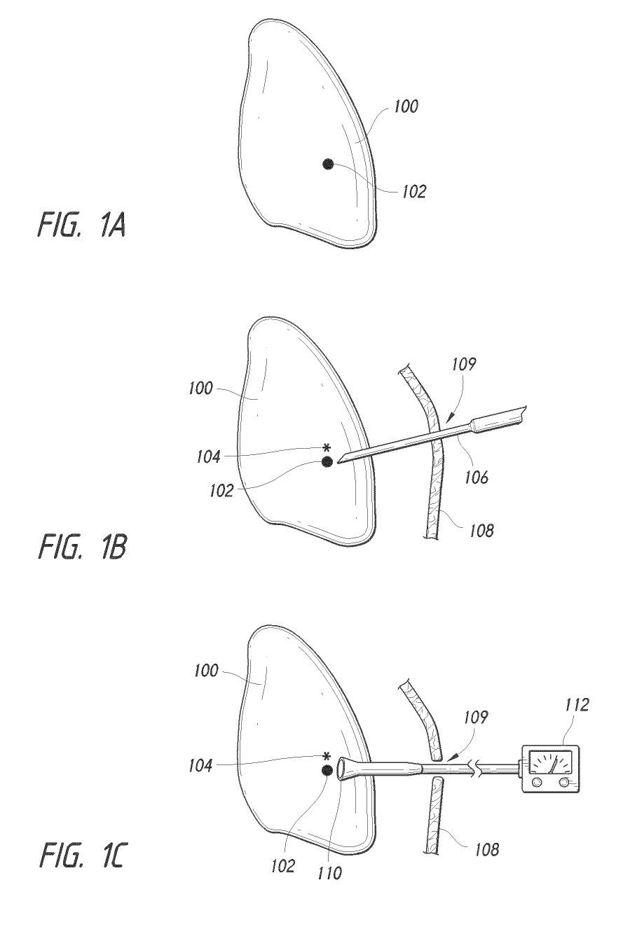

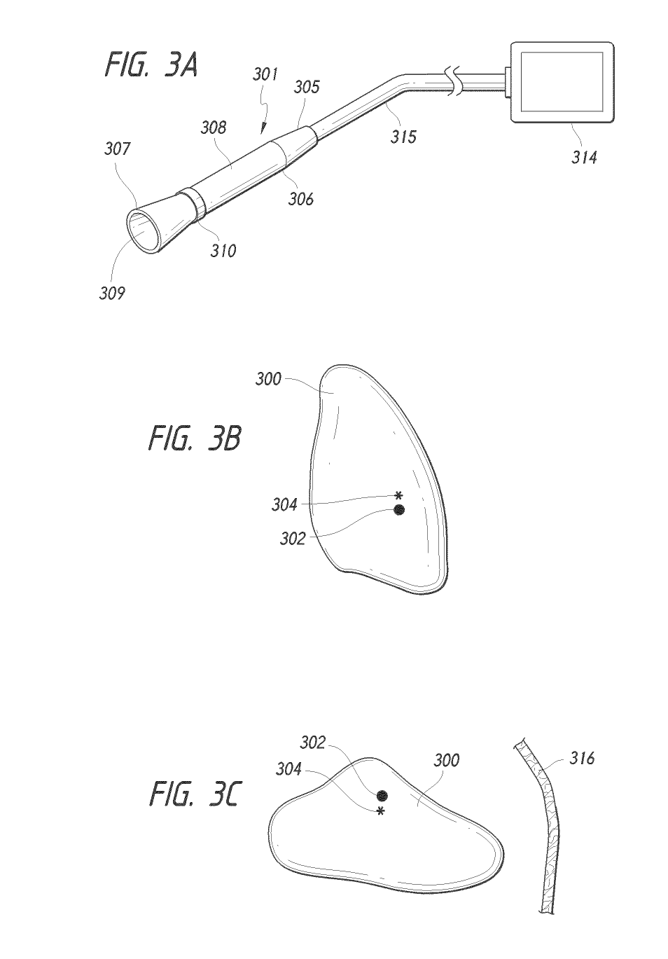

FIGS. 3A-E illustrate an embodiment of a nodule identification and resection device 301 comprising a sensor 310. As illustrated in FIG. 3A, the device 301 comprises a receptacle 306 in fluidic communication with a source of vacuum 314 such as a vacuum pump. The receptacle 306 is configured to aspirate and receive a portion of tissue within itself, and in a preferred configuration is an elongated, hollow cylindrical body 308 with an aperture 309 at a distal end 307. The proximal end 305 is preferably connected to the source of vacuum 314 via a conduit 315, although this connection may be made along any portion of the elongated body 308. Preferably, the sensor 310 is positioned at or near the distal end 307 of the body 308, and may in some embodiments be positioned at or near the aperture 309. The sensor 310 may be of the type described above with reference to FIGS. 1A-C, and is preferably configured to identify a traceable marker 304 that has been implanted into tissue and provided with one or more localization attributes. In a preferred embodiment, the sensor 310 is adapted into the embodiments illustrated in U.S. Pat. Nos. 6,328,689, 6,485,407, 6,491,706, 6,860,847, and 7,731,651, which are hereby incorporated herein by reference in their entireties. In some embodiments, however, the sensor 310 may be separate from the receptacle 306, and could for example be part of a second probe or catheter.

FIG. 3B illustrates a tissue site, here shown as a portion of an inflated lung 300 into which a traceable marker 304 has been implanted in close proximity to a nodule 302. The traceable marker 304 is preferably similar to the embodiments discussed above in relation to FIGS. 1A-C.

FIG. 3C illustrates the lung portion 300 of FIG. 3B in a deflated configuration (typical of a thoracotomy procedure), with the lung portion 300 pulled away from the inner wall 316 of the patient's thoracic wall. In some nodule biopsy or treatment procedures, the lung may need to be deflated before treatment can proceed, and this may make identification of the nodule 302 difficult, especially if the nodule 302 is located deeply within the lung. Preferably, the traceable marker 304 has been implanted prior to deflation of the lung or lung portion. In some embodiments, localization and/or treatment is performed while the lung portion 300 or lung is not deflated.

FIG. 3D illustrates an embodiment of the device 301 being used in part of a procedure to resect the nodule 302 with the aid of the traceable marker 304. The device 301 may for example be introduced via an incision 320 made into the thoracic wall 316 of a patient. The device 301 may also be used as part of a laparoscopic procedure and introduced via a different route, or may in some other embodiments be introduced from within a catheter inserted into a patient's airway.

Here, the receptacle 306, with the aid of the sensor 310, is navigated near the marker 304. The marker 304 may, as described above, be provided with one or more localization attributes that permit the sensor 310 to locate it. As discussed above in FIGS. 1A-C, a navigational aid 312 may be used to aid in the localization of the marker 304, and may in some embodiments be a gauge whose signal varies in relation to the proximity to the marker 304 due to the localization attribute or attributes present on the marker 304.

Once the marker 304 has been located, the aperture 309 is positioned in close proximity to the marker 304, and the vacuum source 314 is activated or placed in fluidic communication with the receptacle 306 so as to suction the marker 304 and the tissue surrounding it (which should include the nodule 302) into the receptacle 306. The potion of tissue within the receptacle 306 may then be resected, for example by cutting the tissue flush with the aperture 309. In some embodiments, a cutting apparatus (not illustrated) integrated with the device 301 may be provided to re sect the tissue within the receptacle 3 06.

FIG. 3E illustrates the lung portion 300 after tissue resection with the device 301. The device 301, having been withdrawn from the patient's thoracic cavity, now contains a tissue section including the lung nodule 302 and the marker 304. At the resection site 322, a seal is preferably made so as to substantially reduce or eliminate the likelihood of air leakage from the remaining lung tissue. In some embodiments, staples or sutures are used to seal the resection site 322. Further devices may also be placed to seal the airways leading to the resection site 322, including valved or obstructing devices as of the types described in U.S. Pat. Nos. 6,293,951, 7,757,692, and 8,021,385, together with U.S. Provisional Application. No. 61/587,621, filed Jan. 17, 2012, and which are hereby incorporated by reference in their entireties.

FIG. 4 illustrates an embodiment of a system 401 that may be used to treat tissue such as a lung nodule 402 with energy, such as microwave radiation, radiofrequency current, or other similar treatment modalities. Preferably, the system 401 comprises a delivery catheter 403 that may be inserted into the working channel of a bronchoscope 405 or other endoscope, the bronchoscope 405 then being inserted into a patient airway 400 and navigated to the nodule 402. In order to navigate the system 401 to the approximate location of the nodule 402, the system 401 can be used and combined with other embodiments described herein, such as those illustrated in FIGS. 1A-C should a traceable marker be used to mark the location of the nodule 402. Of course, navigation using traditional means such as visualization using a bronchoscope or fluoroscopy may be used.

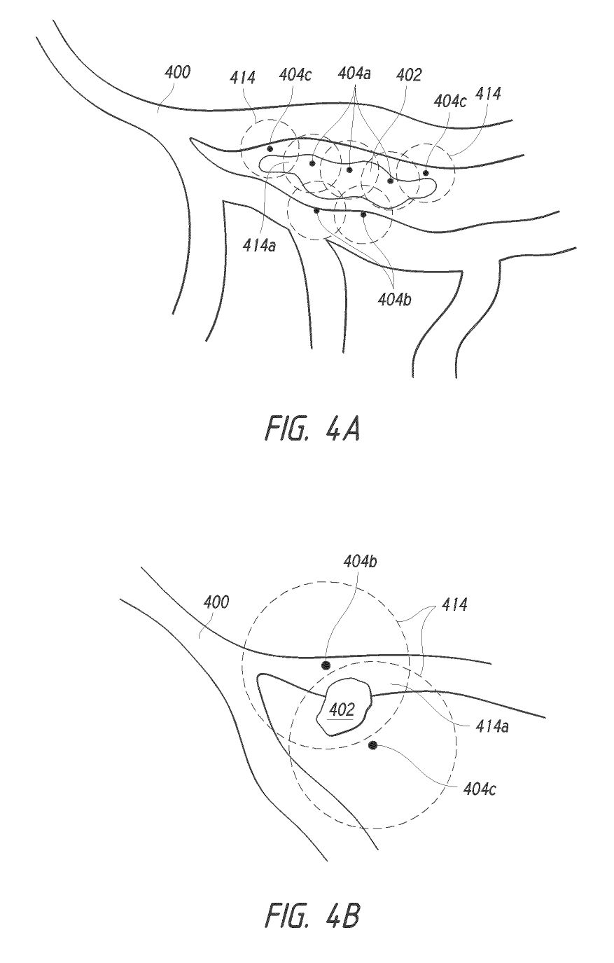

The proximal end 410 of the delivery catheter 403 may be attached to an energy source 412, and the distal tip 408 of the catheter 403 comprises an antenna or other emitter. Examples of antenna tips that may be suitable for use with the system 401 are discussed below in relation to FIGS. 5A-C. In a preferred embodiment, the energy source 412 generates microwaves. The energy emitted from the energy source 412 travels through the delivery catheter 403 and to the antenna on the distal tip 408, from which energy is emitted from the antenna to the surrounding tissue. At least the catheter 403 and antenna are preferably hollow, coaxial, and/or constructed from a material transparent to the wavelength generated in the energy source 412. In some embodiments, the delivery catheter 403, the distal tip 408, and/or the antenna on the distal tip 408 are constructed to form a waveguide configured to channel energy from the energy source 412 to the antenna tip. When using microwaves as an energy source, the waveguide may comprise a hollow, conductive metal conduit. The antenna is preferably provided with one or more openings that allow the energy transmitted from the energy source 412 to exit from the antenna. In some embodiments, the one or more openings may comprise a portion of the antenna where an insulating material does not cover the antenna or where the insulating material has been removed.

The distal tip 408 is placed in close proximity to the nodule 402 such that energy (e.g., microwaves) emitted from the antenna may be used to irradiate, heat, or otherwise treat the nodule 402. Microwaves in some cases may be advantageous in the treatment of lung nodules because they are able to preferentially heat the denser nodule tissue while minimally heating the less-dense surrounding tissue. Thus, a preferred method of treatment may comprise navigating the delivery catheter 403 such that the distal tip 408 is in close proximity to the nodule 402. Activation of the energy source 412, configured here to generate microwaves, then permits the antenna to irradiate the nodule 402 with microwaves. In some embodiments, the distal tip 408, and in particular the antenna, may comprise a protective sheath or covering. In some embodiments, openings may be provided on at least a portion of the antenna, and these may be configured so that the energy emitted by the antenna may pass in a cross-hatch pattern into the nodule 402.

Certain embodiments may use a traceable marker, for example those described above in relation to Figures IA-C, in combination with embodiments of the system 40 I described here. Such a combination may prove advantageous as transmission of the energy emitted from the antenna at the distal tip 408 into the nodule 402 may be facilitated with the presence of the marker. As such, and without wishing to be bound by theory, it is believed that the marker may use less energy, and require less focusing of this energy (e.g., microwaves), compared to a system 40 I that does not use a marker, as the marker absorbs the energy and radiates it as heat to the surrounding tissue in a manner more efficient than if a marker was not used.

The treatment of nodules 402 or other areas of cancerous or diseased tissue may require expanding a zone of treatment beyond the immediate zone of cancerous or diseased tissue, such that sufficient margins are provided around the area to encompass tissue that may not necessarily be showing indicia of disease or cancer. Such margins may encompass tissue that while not necessarily necrotic or cancerous, may, for example, show signs of inflammation. In some embodiments, as illustrated in FIG. 4A, the implantation of multiple markers 404a, 404b, 404c (hereinafter referred to collectively as markers 404) may enable better treatment of the margins surrounding the nodule 402 or other zone of tissue.

The markers 404 can be implanted into the nodule 402 (e.g., inter-nodule markers 404a), into the airway 400 (e.g., intra-airway markers 404b), and/or into tissue outside of the airway 400 and outside the nodule 402 (e.g., intermediary nodules 404c). In some embodiments, each of the markers 404 can be positioned outside of the nodule 402. Placing all of the markers 404 outside the nodule 402 can, in some embodiments, allow for treatment of a nodule 402 without direct physical contact (e.g., piercing) interaction with the nodule 402. In some embodiments, treatment of a nodule 402 without direct physical contact with the nodule can reduce or eliminate the likelihood of release of contents of the nodule 402 (e.g., cancerous cells, infection) to the tissue surrounding the nodule 402. Each of the markers 404 can have an effective zone 414 surrounding the respective markers 404. The effective zones 414 can generally define the extent to which the respective markers 404 effect treatment (e.g., heating, energy application) to the tissue surrounding the markers 404. The effective zones can 414 can have a generally spherical shape (e.g., for generally spherical markers 404). In some embodiments, the effective zones 414 have oblong or other shapes. According to some variants, the effective zone 414 of a given marker 404 generally follows the shape of that marker 404. The size of an effective zone 414 of a given marker 404 can vary depending on a one or more parameters. For example, in some embodiments, the size of an effective zone 414a can depend, in part, on the size of the associated marker 404. In some embodiments, the size of an effective zone 4 I 4a can depend on the amount of power emit by or into the associated marker 404. In some embodiments, the size of an effective zone 414a can depend on the nature of the tissue into which the marker 404 is deployed (e.g., the effective zone 414a can vary depending on the density and/or resistivity of the tissue into which the marker 404 is deployed).

The treatment efficacy (e.g., the extent to which the marker heats or otherwise treats the surrounding tissue) of each marker 404 within the effective zones 414 at points in the zones 414 can, in some embodiments, diminish at distances further from the markers 404 (e.g., radially-outward points in the effective zones 414 in the case of spherical zones 414). The markers 404 can be distributed such that their respective effective zones 414 overlap in overlap zones 414a in the tissue being treated. In some embodiments, the overlap zones 414a can realize higher treatment efficacy (e.g., greater heating, higher energy application) than the equivalent points in the treatment zones would realize without overlap.

In some embodiments, the treatment efficacy of each individual marker 404 can be low enough such that one or more of the individual markers 404 would not, themselves, effect treatment of the tissue surrounding the marker 404. For example, one or more of the individual markers 404 can be configured to emit heat or other energy to the surrounding tissue at a level that would not, per individual marker 404, damage or otherwise effect change in the surrounding tissue. In some such embodiments, the overlap zones 414a can realize cumulative treatment efficacy that is high enough to effect treatment of the tissue within the overlap zones 414a. For example, as illustrated in 4B, two or more markers 404 can be positioned into or near a nodule 402 such that the overlap zone 414a created by the two or more markers 404 completely envelopes the nodule 402.

In some embodiments, application of energy such as, for example, microwave or radiofrequency energy, could selectively heat or thermoablate a zone of treatment that is equal to or greater than the margins around the nodule 402 that encompass tissue that is either diseased or cancerous, or likely to become diseased or cancerous. In some embodiments, the use of multiple markers 404 can facilitate the creation of a customized zone of treatment that more closely maps to the margins around the nodule 402 that encompass tissue that requires treatment.

FIGS. 5A-C illustrate embodiments of antenna tips that may be used for treating a lung nodule or other site, for example with energy such as microwave radiation or other similar treatment modalities. These antenna tips, for example, may be used in embodiments such as those described above in FIG. 4. Figure SA illustrates an embodiment of an antenna tip 508 on the distal tip 506 of a catheter 505. As described in FIG. 4, in some embodiments the catheter 505 may be inserted into a bronchoscope or other endoscope (not illustrated). Here, the distal end of the catheter 506 is deployed so as to circumscribe or loop around all or a part of a region of tissue such as a lung nodule 502. In some embodiments, the distal end of the catheter 506 may be inserted into the nodule 502 so as to circumscribe an interior portion thereof. The antenna 508 and/or energy used are preferably designed so as to emit or direct the energy inwards toward the region of circumscribed tissue, and the antenna S08 may incorporate adaptations such as holes or waveguides that preferentially focus the radiation.

Figure SB illustrates another embodiment with dual or multiple antenna tips S08, S09. Here, antenna tips S08, S09 are inserted in proximity to a site of interest such as a nodule S02. The antenna tips S08, S09 may, as illustrated here, be navigated to the nodule S02 via one catheter SOS. Multiple catheters, needles, or other endoscopic apparatuses may also be used alone or in combination to place the antenna tips S08, S09 in proximity to the nodule S02. In a dual or multiple antenna tip arrangement, the respective antenna tips S08, S09 are preferably arranged so as to direct their energy inwards. Accordingly, when these tips S08, S09 are placed in proximity to the nodule S02, energy such as microwave radiation or radiofrequency current may be emitted or directed toward the nodule S02 so as to heat or irradiate it.

In some embodiments using bipolar high frequency (e.g., radiofrequency) current, a system comprising at least two antenna tips S08, S09 functioning as electrodes may be used to treat the nodule S02. Here, positioning the antenna tips S08, S09 on opposite sides of the nodule S02 permits an electric field to flow between the two antenna tips S08, S09, thus causing heating and/or thermoablation of the nodule S02 and intervening tissue. Such a treatment modality may be advantageous if, for example, a nodule S02 is located between two branches of an airway. In such a situation, the antenna tip S08 may be advanced along one airway and positioned in proximity to one side of the nodule S02, and the antenna tip S09 may be advanced along the other airway and positioned similarly along another side of the nodule S02. When activated, the antenna tips S08, S09 would then cause heating of the nodule S02.

Figure SC illustrates an embodiment of a pinpoint antenna tip S08. As with the preceding figures, the distal tip S06 of a catheter SOS comprises an antenna tip S08. While this embodiment may function in a similar fashion as the other embodiments illustrated in FIG. 4 and Figures SA-B to irradiate or heat tissue with radiation (e.g., microwave radiation), here the antenna tip S08 is configured to function as a pinpoint source of radiation. As such, the antenna tip S08 preferably is inserted into tissue, such as a nodule S02, and upon activation of an energy source, such as a microwave generator, for example, radiation emanates outward from the antenna tip 508 so as to heat or irradiate surrounding tissue.

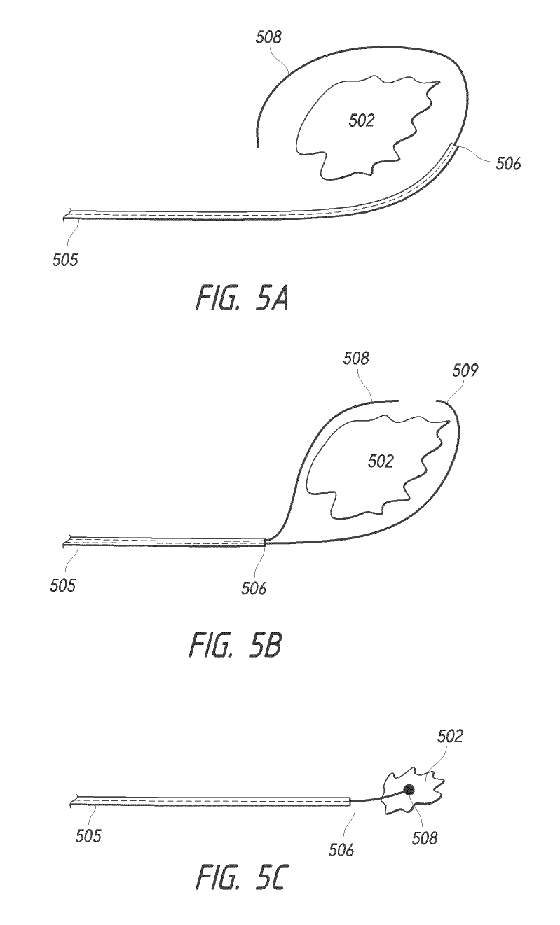

FIG. 6 illustrates an embodiment where a traceable marker 604 is used in conjunction with magnetic coupling to heat a region of tissue such as a nodule 602. The marker 604, which can be the traceable marker described above in Figures IA-C, is preferably implanted into or near the nodule 602. A catheter 605 may then be introduced into an airway 600 so as to position a distal end 607 of the catheter 605 proximate the nodule 602. The distal end 607 comprises a magnetic probe 608, which in some embodiments comprises a loop of wire. Electrical current flows through the probe 608 and, when in proximity to the marker 604, the two respective parts become inductively or magnetically coupled, thereby causing the marker 604 to heat up and deliver a treatment modality such as thermal therapy or thermoablation to the nodule 602 and/or the surrounding tissue. In such embodiments, the marker 604 is made from a material that can be heated using magnetic or inductive coupling, and may comprise metals, especially ferromagnetic metals, such as stainless steel or iron, for example. In some embodiments, the marker 604 comprises a reservoir filled with iron particles (such as microparticles or filings) suspended or mixed in a liquid medium such as, for example, a saline solution.

Although FIG. 6 illustrates the catheter 605 being introduced into the airway 600 to bring the probe 608 in close proximity to the marker 604, the probe 608 may be introduced by other means, including laparoscopic probes or any other suitable means. Additionally, the magnetic heating of the marker 604 may be adjusted based on several factors, including the amount and frequency of the current passed through the probe 608, and the distance between the probe 608 and the marker 604. In some embodiments, the probe 608 may not need to be introduced into the patient's airway or tissue, and may be placed over the patient's skin.

In some embodiments, it is not necessary to use a probe 608 to induce magnetic heating of the marker 604. For example, the marker 604 may be activated or heated using magnetic coupling via a Magnetic Resonance Imaging (“MRI”) device, which may be advantageous as such devices are present in many hospitals and other patient care settings. In such embodiments, the magnetic field and/or field frequency applied by the MRI device and/or the marker 604 is configured such that the marker 604 activates or heats without significant migration when under the influence of the magnetic field. Without wishing to be bound by theory, it is believed that there is a linear relationship between the magnetic field applied and the resulting heating of the marker 604, and as such the field may be tailored (alone or in combination with other variables) to achieve appropriate heating of the marker 604. In some embodiments, an applied magnetic field in the range of 1.5-3 T, and in particular IST, has been found sufficient to induce heating.

In some embodiments, the marker 604 may be made MRI-compatible by having it substantially respond only to magnetic fields stronger than those generated by an MRI device. For example, most MRI devices function in a range between 1.5-3 T, and a marker 604 may be designed so as to substantially respond to a magnetic field greater than 4 T. The marker 604 may then be used in conjunction with a device capable of generating such a field for magnetic heating of the nodule 602 while still being MRI-compatible.

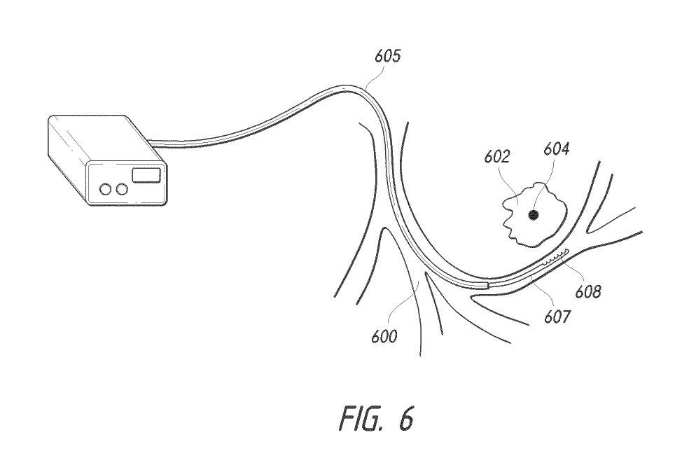

FIGS. 7A-B illustrate an embodiment of a traceable marker 704 comprising a tail 706, where the tail 706 is attached at its distal end to the marker 704. In a preferred embodiment illustrated in FIG. 7A, the tail 706 is electrically conductive and in electrical communication with the marker 704, and may function as an auxiliary power lead to the marker 704. It may be advantageous to such embodiments when accessing areas of tissue, such as a nodule 702, that are difficult to access. For example, although a marker 704 may be implanted via a bronchoscope, navigating to the site of the marker 704 at a subsequent time (e.g., after biopsying the nodule 702 indicates that cancerous tissue is likely to be present) may be challenging, especially in smaller diameter peripheral lung passages where visual navigation may be limited or impossible. As such, implantation of the marker 704 into the nodule 702 is preferably performed so that the tail 706 extends in a proximal direction (e.g., toward the larger airways leading toward the trachea). Similar embodiments are described in

U.S. patent application Ser. No. 13/778,008, entitled PULMONARY NODULE ACCESS DEVICES AND METHODS OF USING THE SAME, filed Feb. 26, 2013 and hereby incorporated by reference herein in its entirety, and which may be used in conjunction with the embodiments described herein.

Some embodiments may also use an anchoring mechanism positioned along the tail 706, and in particular at its distal end. This anchoring mechanism may be used to secure the tail 706 to tissue (e.g., a portion of an airway). In some embodiments, all or part of the tail 706 is radioopaque, which may be beneficial when used in conjunction with fluoroscopy techniques. Together with embodiments such as those illustrated in FIG. 4, the tail 706 may be connected to an energy source such as a microwave generator. The tail 706 may thus be used to focus energy at the core of the nodule 702 into which the marker 704 has been inserted.

FIG. 7B illustrates how a proximal end of the tail 706 may be connected to a catheter 708 or other device that may be used to supply power to the marker 704. Preferably, the catheter 708 is introduced into a patient's airway via a working channel 712 of a bronchoscope 710 or other endoscope. The catheter 708 preferably comprises a connection element connecting to the tail 706 and that is in electrical communication with a source of power so as to supply power to the marker 704. Examples of such connection elements include plug and socket connectors, jacks, clamps, and so forth. In some embodiments, the power supplied to the marker 704 may be used to power a heating element in the marker 704 or to otherwise initiate heating therapy, thermoablation, or some other treatment modality. For example, a resistive heating element incorporated into the marker 704 and powered via the tail 706 may be used.

Additional embodiments may use the power supplied to the marker 704 for electrical therapy. The power may also be used to power sensors or other devices integrated into the marker 704. For example, the power may be used to apply an electrical field to a marker 704 comprising a piezoelectric material. In some embodiments, applying an electrical field to a marker 704 comprising a piezoelectric material can cause the marker 704 to emit an ultrasonic wave to the tissue surrounding the marker 704. The ultrasonic wave can be used to treat (e.g., heat) the tissue surrounding the marker 704. In other embodiments, the power supplied to the marker 704 may be used to trigger treatment modalities such as the release of agents on or within the marker 704. The electrical power supplied may also be used to induce electroporation of the cells in the nodule 702 so as to increase their permeability to chemicals or other therapeutic agents. Thus, selective therapy of the nodule 702 may be achieved. In some embodiments, electrical therapy from power supplied to the marker 704 may be used in addition, in combination, or as an alternative to other treatment modalities.

In some embodiments, the marker 704 may be configured to cool and/or heat tissue surrounding the marker 704 through use of the Peltier effect. For example. the marker 704 can be constructed from at least two different materials (e.g., two metals) having a junction through which an electric current is directed. In some embodiments, heat is absorbed on one side of the junction and heat is generated on the other side of the junction. A heat sink (e.g., a conductive wire or other structure) can be coupled to the heat generating side of the junction to dissipate the generated heat. In some embodiments, dissipation of the heat from the heat generating side of the junction and heat absorption from the opposite side of the junction can cool the tissue surrounding the marker 704. In some embodiments, the heat generated from the heat generating side of the marker 704 can be used to heat and/or thermoablate a portion of tissue surrounding the marker 704.

Some embodiments may also provide for injecting or releasing an enhancement substance such as an electrically or thermally conductive fluid or gel in the tissue and space around the marker 704. Such an enhancement substance may for example be released from the marker 704, or be injected (for example, by using a catheter or needle) around the marker 704. The use of an enhancement substance may allow the treatment modality used in conjunction with the marker 704 to affect a greater area of tissue near the marker 704 and specifically the nodule 702. Such an effect may enhance treatment of the tissue margins surrounding the nodule 702, which may be cancerous or pre-cancerous but not yet identifiable as such.

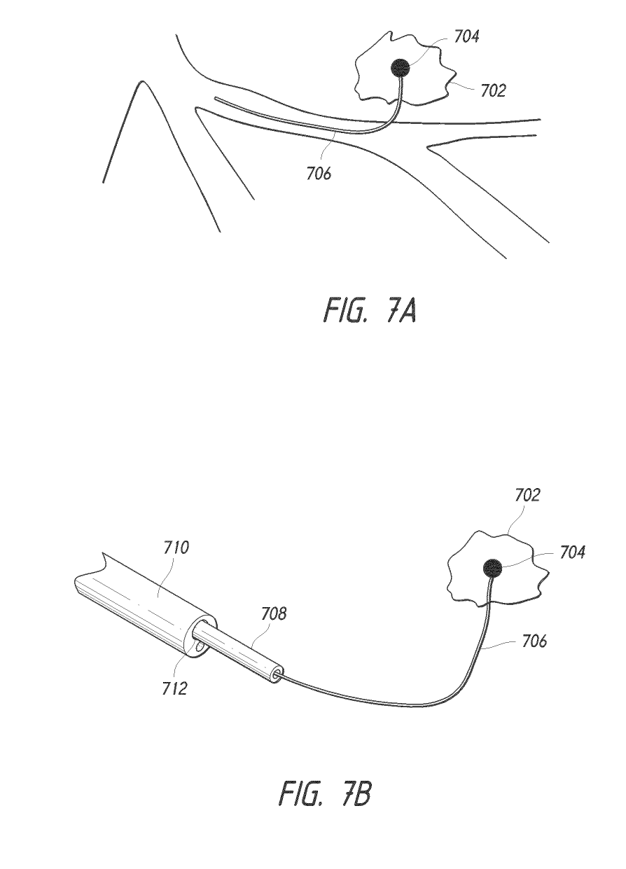

FIG. 8 illustrates an embodiment of a marker 804 comprising a secondary module 808. In this embodiment, the marker 804, being preferably implanted into tissue such as a lung nodule, is connected via an auxiliary power lead or tail 806 to a secondary module 808. In a preferred embodiment, the secondary module 808 comprises a battery, capacitor, or other power storage or generation module, and may also comprise a controller. The secondary module 808 may be used to provide power to the marker 804, in a manner similar to the embodiments described in FIGS. 7A-B, except that accessing the marker 804 via, for example, a catheter may not be necessary to provide power, as the secondary module 808 may be used to provide power in lieu or in addition to a connection via a catheter. In some embodiments, the marker 804 can be powered or otherwise actuated without the use of a catheter or secondary module 808. For example, the marker 804, itself, can comprise a power source (e.g., a battery, capacitor, or other power storage device or component insider and/or coupled with the marker 804). The controller, in addition to controlling the power (including voltage and current) delivered to the marker 804, may also comprise a control unit that can run programs and/or select therapy or treatment regimes via heating or other action on the marker 804. In some embodiments, the controller may also comprise a wireless receiver that can be externally activated, or that may receive instructions or transmit information to and from the secondary module 808 and/or the marker 804. Preferably, the secondary module 804 is miniaturized, and may be implanted subcutaneously or within an air passage.

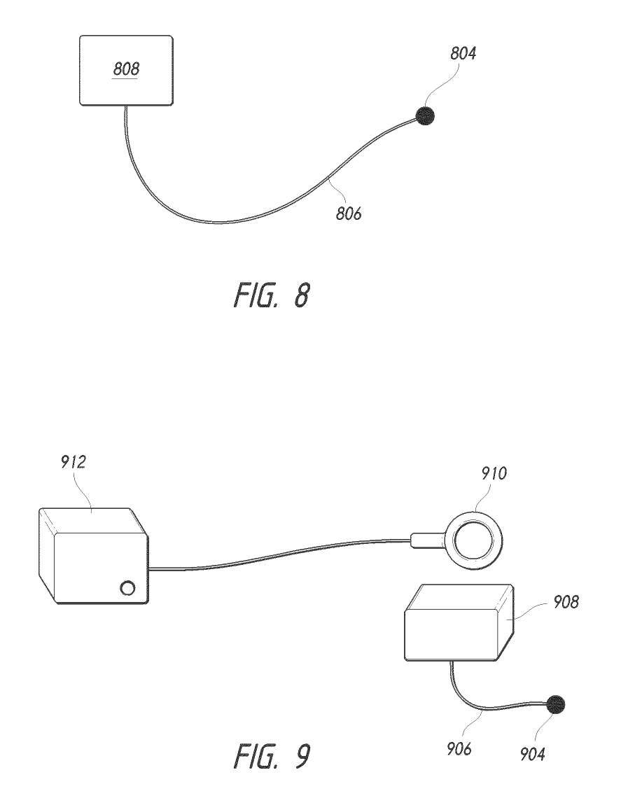

FIG. 9 illustrates an embodiment of a marker 904 comprising a secondary module 908 being activated and/or controlled by a wand 910. Here, the marker 904 and secondary module 908 may be similar to the embodiment illustrated in FIG. 8, with at least the secondary module 908 being preferably implanted subcutaneously. The marker 904 is connected to the secondary module 908 via a tail or auxiliary power lead 906. The wand 910 may be brought into close proximity or waved over the approximate site of the secondary module 908, and may thus be used to activate or charge the secondary module 908 wirelessly. In some embodiments, the secondary module 908 may comprise a controller and power source, as described above. Preferably, the wand 910 is connected to a master control unit 912.

The secondary module 908 may comprise a wireless charging module. In one embodiment, the wireless charging module may comprise a passive coil that is activated when the wand 910 is passed over it. The wand 910 may be connected to a charging mechanism present in the master control unit 912, and which may comprise a RF or high frequency generator that can charge the battery or other power storage module present in the secondary module 908 via the wand 910. In some embodiments, the wand 910 may also be used to activate and/or control the secondary module 908 and/or the marker 904 so as to activate therapy (e.g., heating), transmit data, and so forth.

Although this invention has been disclosed in the context of certain embodiments and examples, those skilled in the art will understand that the present invention extends beyond the specifically disclosed embodiments to other alternative embodiments and/or uses of the invention and obvious modifications and equivalents thereof. In addition, while several variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes or embodiments of the disclosed invention. Thus, it is intended that the scope of the present invention herein disclosed should not be limited by the particular disclosed embodiments described above.

1. (canceled)

2. A system for the treatment of a region of tissue, the system comprising:
a catheter configured to be inserted within a patient's airway, wherein the catheter comprises an antenna at a distal end of the catheter;
a microwave generator configured to be connected to the catheter so as to emit microwave radiation from the antenna.

3. The system of claim 2, further comprising a bronchoscope, wherein the catheter is configured for insertion into a working channel of the bronchoscope.

4. The system of claim 2, wherein the antenna tip is configured to circumscribe an exterior perimeter of a lung nodule.

5. The system of claim 2, wherein the antenna tip is configured to be inserted into a lung nodule.

6. The system of claim 2, further comprising a second antenna tip at the distal end of the catheter.

7. A method of repeatedly locating a position in a lung, the method comprising: navigating to the position within the lung;
implanting a traceable marker into the position, wherein the traceable marker comprises at least one localization attribute;
locating the traceable marker based on the at least one localization attribute.

8. The method of claim 7, further comprising implanting a second traceable marker near the position within the lung.

9. The method of claim 7, wherein the step of navigating comprises using a bronchoscope.

10. The method of claim 7, wherein the traceable marker is implanted via a catheter or needle inserted into a working channel in the bronchoscope.

11. The method of claim 7, further comprising biopsying a tissue sample at or near the position in the lung.

12. The method of claim 7, wherein the at least one localization attribute is selected from the group consisting of: radioopacity localization, magnetism localization, radioactivity localization, and visual localization.

13. The method of claim 7, wherein the step of locating the traceable marker comprises using a localization device.

14. The method of claim 13, wherein the localization device comprises a magnetic sensor.

15. The method of claim 7, further comprising applying treatment to the position in the lung,

16. The method of claim 15, wherein the treatment is repeatedly applied to the position in the lung by repeatedly locating the traceable marker.

17. The method of claim 15, wherein the treatment comprises resecting lung tissue near or at the position in the lung.

18. The method of claim 17, wherein the step of resecting lung tissue comprises positioning a receptacle in fluid communication with a source of vacuum near the position in the lung, activating the source of vacuum, suctioning lung tissue into the receptacle, and severing the tissue within the receptacle from the remainder of the lung.

19. The method of claim 15, wherein the treatment comprises applying microwave radiation.

20. The method of claim 19, wherein the microwave radiation is applied via an antenna tip connected to a source of microwave radiation, the antenna tip being inserted into a catheter.

21. The method of any of claims 15, wherein the treatment comprises applying heat.

22. The method of claim 21, wherein the heating is applied using a magnetic field acting upon the traceable marker.

23. The method of claim 22, wherein the magnetic field is applied via a probe positioned near the traceable marker.

24. The method of claim 22, wherein the magnetic field is applied using an MM device.

25. The method of claim 15, wherein the treatment comprises applying electrical treatment,

26. The method of claim 15, wherein the traceable marker comprises a treatment modality.

27. The method of claim 26, wherein the traceable marker is configured to release one or more therapeutic agents.

28. The method of claim 15, wherein the traceable marker comprises a power source,

29. The method of claim 28, wherein the power source is a battery attached to the traceable marker.

30. The method of claim 28, wherein the power source is attached to the traceable marker via a wire.
